(19) United States
(12) Patent Application Publication
(10) Pub. No.: US 2025/0058016 A1
(43) Pub. Date: Feb. 20, 2025

(54) PROTEASE COMPOSITIONS AND METHODS OF USE

(71) Applicant: Swiss-American CDMO, LLC, Carrollton, TX (US)

(72) Inventors: Priya Niranjan, Carrollton, TX (US); Komel V. Grover, Carrollton, TX (US)

(21) Appl. No.: 18/233,680

(22) Filed: Aug. 14, 2023

(51) Int. Cl. A61L 26/00

(52) U.S. Cl. CPC A61L 26/0057; A61L 2300/254; A61L 2430/34

(57) ABSTRACT

In an embodiment, the present disclosure pertains to a method to promote tissue repair and wound healing. In general, the method includes applying a protease composition to an area of skin, modulating, by the protease composition, at least one of a wound-related protein or an inflammation-related protein, and treating the area of skin for a prescribed period of time. In some embodiments, the protease composition includes one or more proteases. In an additional embodiment, the present disclosure pertains to a kit to promote tissue repair and wound healing. In general, the kit includes, inter alia, a wound-related protein or inflammation-related protein modulating protease composition comprising one or more proteases. In some embodiments, each of the one or more proteases is in a solution at a concentration of 10⁻⁸ to 2% (w/v).

### TECHNICAL FIELD

The present disclosure relates generally to protease compositions and more particularly, but not by way of limitation, to protease compositions and methods of use.

### BACKGROUND

This section provides background information to facilitate a better understanding of the various aspects of the disclosure. It should be understood that the statements in this section of this document are to be read in this light, and not as admissions of prior art.

Humans are capable of replacing injured skin and cells by repairing tissue damage, and typically, the defect is initially replaced by a fibrous scar, which is later remodeled. During the transitional coagulation stage there is temporary wound closure through the formation of a blood clot containing thrombocytes and fibrin. The a-granules in the thrombocytes release various growth factors, such as, for example, platelet-derived growth factor (PDGF), insulin-like growth factor I (IGF-I), transforming growth factor alpha (TGF-α) and epidermal growth factor (EGF). TGF-α and tumor necrosis factor α (TNFα) are secreted from vascular endothelial cells, keratinocytes and fibroblasts inducing the inflammatory stage. This stage lasts only a few days under normal conditions. Granulocytes and macrophages that are present in the wound continuously produce cytokines and proteases which degrade injured or denatured extracellular matrixes (ECMs). Macrophages continue secreting inflammatory and pro-inflammatory cytokines maintaining the inflammatory response until down-regulation and movement into the next stage of healing occurs.

In wounds with intact skin, but having underlying tissue trauma, such as, for example, sports injury or hematoma, although the skin is not replaced, the body nevertheless undergoes an inflammatory response and must remove dead or injured tissue and cells. Following the inflammatory stage, vascular angiogenesis with capillary formation and development of granulation tissue occurs during the subsequent granulation stage. In this stage, predominantly collagen replaces the basic matrix made up of fibrin, fibronectin and hyaluronic acid.

Common characteristics of various healing types are the consecutive closure of the wound and the simultaneous replacement of the injured tissues. While most wound portions are filled by connective cell material some tissues, such as, for example, brain, nerves, connective tissue and bones are replaced by other appropriate and adequate material.

Wound healing is a complicated process. Generally, acute wounds are those that heal rapidly and proceed through the inflammatory, proliferation and remodeling phases of wound healing. However, chronic wounds often become senescent in the inflammatory or proliferation stages and cannot progress to closure. In addition to implementing treatment regimens that address the etiology and symptoms, clinicians prepare the wound for healing by removing dead tissue, reducing the bacterial bioburden, decreasing edema, managing exudate and enhancing angiogenesis. However, even though the wound bed may appear ready to heal, the microenvironment may be out of balance thus impeding healing and frustrating both the patient and the clinician.

In general, the microenvironment of the wound is a web of intertwining, cells, proteins, enzymes, fluids and pathways, which perform specific functions that normally are tightly regulated. In wounds that chronically fail to heal, however, the microenvironment has become deregulated with various components being over-expressed, under-expressed, inactive or ineffective. Specific protein comparisons between acute and chronic wounds revealed that chronic wounds generally have excessive levels of matrix metalloproteinases (MMPs), high levels of inflammatory cytokines TNFα, interleukin-1 (IL-1) and interleukin-6 (IL-6), and minimal levels of tissue inhibitor metalloprotainases (TIMPs) and growth factors, such as, for example, transforming growth factor beta (TGF-β) and EGF. To complicate matters, activated inflammatory cells stimulate MMP production and suppress TIMPs by secreting TNFα and interleukin-1β (IL-1β), which impair the healing process via increased inflammation and degradation of ECM components, growth factors and receptors contributing to multiple negative feedback loops preventing wound closure.

Promoting, returning to and maintaining a normal wound microenvironment can be difficult task. Past use of isolated molecules or compounds to modify the healing process have been met with limited success. These limitations may be due to one molecule trying to modify the entire wound environment in a narrowly selected function, or due to the duplicity of multiple alternative pathways or both. Additionally, hostile chronic wound environments degrade exogenously applied growth factors as easily as the intrinsic ones, resulting in little clinical or molecular impact.

An alternative way to return to a more normal wound microenvironment is to modulate the activity of proteins such as MMPs and pro-inflammatory cytokines, which helps promote the hostile environment when in excess. MMPs are normally prevented from destroying too much extracellular matrix (ECM) and tissue by the action of TIMPs that form very specific inhibitory complexes with the MMPs. However, in chronic wounds the ratio of MMP to TIMP is high, such that most of the MMPs are uninhibited. In fact, with elevated MMP levels, the TIMP molecules themselves can be hydrolyzed.

As such, additional approaches are needed to modulate the action and levels of specific proteins in order to promote tissue repair and wound healing, as well as to improve the overall environment of a tissue and its surroundings during the healing process.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a method to promote tissue repair and wound healing. In general, the method includes applying a protease composition to an area of skin, modulating, by the protease composition, at least one of a wound-related protein or an inflammation-related protein, and treating the area of skin for a prescribed period of time. In some embodiments, the protease composition includes one or more proteases.

In some embodiments, the prescribed period of time is in a range of 6 to 72 hours. In some embodiments, the one or more proteases can include, without limitation, acrosin, actinidain, ananain, asclepain, aspergillopepsin I, bacterial leucyl aminopeptidase, brachyurin, bromelain, calpain, carboxypeptidase A, caricain, cathepsin, chymopapain, chymosin, chymotrypsin, complement subcomponent C1r, cytosol aminopeptidase, DD-transpeptidase, dipeptidyl peptidase, deuterolysin, elastase, enteropeptidase, ficain, fragilysin, glycyl endopeptidase, hypodermin, ingensin, kallikrein, kininase, L-peptidase, methionine aminopeptidase, papain, pepsin, peptidyl-glycinamidase, plasmin, proproteinase, semenogelase, streptogrisin, subtilisin, thrombin and combinations thereof. In some embodiments, the one or more proteases is in a solution at a concentration of 10⁻⁸ to 2% (w/v).

In some embodiments, the method further includes applying a second protease composition having at least one protease to the area of skin, modulating, by the second protease composition, the at least one of the wound-related protein or the inflammation-related protein, and treating the area of skin for a second prescribed period of time. In some embodiments, the second prescribed period of time is in a range of 6 to 72 hours. In some embodiments, the at least one protease can include, without limitation, acrosin, actinidain, ananain, asclepain, aspergillopepsin I, bacterial leucyl aminopeptidase, brachyurin, bromelain, calpain, carboxypeptidase A, caricain, cathepsin, chymopapain, chymosin, chymotrypsin, complement subcomponent C1r, cytosol aminopeptidase, DD-transpeptidase, dipeptidyl peptidase, deuterolysin, elastase, enteropeptidase, ficain, fragilysin, glycyl endopeptidase, hypodermin, ingensin, kallikrein, kininase, L-peptidase, methionine aminopeptidase, papain, pepsin, peptidyl-glycinamidase, plasmin, proproteinase, semenogelase, streptogrisin, subtilisin, thrombin and combinations thereof. In some embodiments, the at least one protease is in a solution at a concentration of 10⁻⁸ to 2% (w/v).

In some embodiments, the method includes applying a third protease composition having at least one protease to the area of skin, modulating, by the third protease composition, the at least one of the wound-related protein or the inflammation-related protein, and treating the area of skin for a third prescribed period of time. In some embodiments, the third prescribed period of time is in a range of 6 to 72 hours. In some embodiments, the at least one protease can include, without limitation, acrosin, actinidain, ananain, asclepain, aspergillopepsin I, bacterial leucyl aminopeptidase, brachyurin, bromelain, calpain, carboxypeptidase A, caricain, cathepsin, chymopapain, chymosin, chymotrypsin, complement subcomponent C1r, cytosol aminopeptidase, DD-transpeptidase, dipeptidyl peptidase, deuterolysin, elastase, enteropeptidase, ficain, fragilysin, glycyl endopeptidase, hypodermin, ingensin, kallikrein, kininase, L-peptidase, methionine aminopeptidase, papain, pepsin, peptidyl-glycinamidase, plasmin, proproteinase, semenogelase, streptogrisin, subtilisin, thrombin and combinations thereof. In some embodiments, the at least one protease is in a solution at a concentration of 10⁻⁸ to 2% (w/v).

In some embodiments, the method further includes applying a fourth protease composition having at least one protease to the area of skin, modulating, by the fourth protease composition, the at least one of the wound-related protein or the inflammation-related protein, and treating the area of skin for a fourth prescribed period of time. In some embodiments, the fourth prescribed period of time is in a range of 6 to 72 hours. In some embodiments, the at least one protease can include, without limitation, acrosin, actinidain, ananain, asclepain, aspergillopepsin I, bacterial leucyl aminopeptidase, brachyurin, bromelain, calpain, carboxypeptidase A, caricain, cathepsin, chymopapain, chymosin, chymotrypsin, complement subcomponent C1r, cytosol aminopeptidase, DD-transpeptidase, dipeptidyl peptidase, deuterolysin, elastase, enteropeptidase, ficain, fragilysin, glycyl endopeptidase, hypodermin, ingensin, kallikrein, kininase, L-peptidase, methionine aminopeptidase, papain, pepsin, peptidyl-glycinamidase, plasmin, proproteinase, semenogelase, streptogrisin, subtilisin, thrombin and combinations thereof. In some embodiments, the at least one protease is in a solution at a concentration of 10⁻⁸ to 2% (w/v).

In some embodiments, the method further includes reducing itch-related symptoms at the area of skin. In some embodiments, the wound-related protein can include, without limitation, matrix metalloproteinases (MMPs), matrix metalloproteinase-2 (MMP-2), matrix metalloproteinase-3 (MMP-3), matrix metalloproteinase-9 (MMP-9), tissue inhibitor metalloprotainases (TIMPs), TIMP metallopeptidase inhibitor 1 (TIMP-1), TIMP metallopeptidase inhibitor 2 (TIMP-2), transforming growth factor alpha (TGF-α), interleukins, interleukin-1β (IL-β), interleukin-6 (IL-6), interleukin-10 (IL-10), growth factors, platelet-derived growth factor-AB (PDGF-AB), insulin-like growth factor I (IGF-I), transforming growth factor beta (TGF-β), epidermal growth factor (EGF), fibroblast growth factor (FGF) basic, granulocyte colony stimulating factor (G-CSF), granulocyte-macrophage colony-stimulating factor (GM-CSF), vascular endothelial growth factor (VEGF), interferons, interferon-α (IFNα), interferon-γ (IFNγ), C-reactive protein (CRP), macrophage inflammatory proteins (MIPs), macrophage inflammatory protein-1 alpha (MIP-1α), macrophage inflammatory protein-1 beta (MIP-1β), macrophage inflammatory protein-2 (MIP2) and combinations thereof. In some embodiments, the inflammation-related protein can include, without limitation, TNFα, interleukins, such as, for example, IL-β, IL-6, IL-10, serum amyloid A, fibrinogen, interferons, IFNα, IFNγ, CRP, MIPs, MIP-1α, MIP-1β, MIP2, MMPs, MMP-2, MMP-3, MMP-9 and combinations thereof.

In an additional embodiment, the present disclosure pertains to a kit to promote tissue repair and wound healing. In general, a wound-related protein or inflammation-related protein modulating protease composition comprising one or more proteases and a wound covering material. In some embodiments, each of the one or more proteases is in a solution at a concentration of 10⁻⁸ to 2% (w/v). In some embodiments, the wound-related protein can include, without limitation, MMPs, MMP-2, MMP-3, MMP-9, TIMPs, TIMP TIMP-1, TIMP-2, TGF-α, interleukins, IL-β, IL-6, IL-10, growth factors, PDGF-AB, IGF-I, TGF-β, EGF, FGF basic, G-CSF, GM-CSF, VEGF, interferons, IFNα, IFNγ, CRP, MIPs, MIP-1α, MIP-1β, MIP2 and combinations thereof. In some embodiments, the inflammation-related protein can include, without limitation, TNFα, interleukins, such as, for example, IL-β, IL-6, IL-10, serum amyloid A, fibrinogen, interferons, IFNα, IFNγ, CRP, MIPs, MIP-1α, MIP-1β, MIP2, MMPs, MMP-2, MMP-3, MMP-9 and combinations thereof.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

In various embodiments, the present disclosure provides protease compositions and methods of use for promoting tissue health and repair by modulating the protein profile within the tissue or its surrounding environment. Tissue can become damaged as a result of external forces, such as, for example, trauma or injury, which in turn can lead to wounds or inflammation. Alternately, tissues can become damaged as a result of internal forces, such as, for example, disease and genetic factors. Repair of tissue damage is a complex process which requires control of the environment at the point of damage and the surrounding areas. An aspect of the repair process requires the modulation of the protein profile in and around the damaged tissue. This means that the levels or activities of certain proteins must be modulated, that is, increased or decreased, in order to create an environment that promotes the repair process.

The present disclosure provides protease compositions and methods of use that modulate the activity of wound-related proteins such as matrix metalloproteinases (MMPs), cytokines and growth factors, thereby promoting wound healing. Wound-related proteins can include, but are not limited to: (1) MMPs, such as, for example, matrix metalloproteinase-2 (MMP-2), matrix metalloproteinase-3 (MMP-3), matrix metalloproteinase-9 (MMP-9); (2) tissue inhibitor metalloprotainases (TIMPs), such as, for example, TIMP metallopeptidase inhibitor 1 (TIMP-1) and TIMP metallopeptidase inhibitor 2 (TIMP-2); (3) transforming growth factor alpha (TGF-α); (4) interleukins, such as, for example, interleukin-1β (IL-β), interleukin-6 (IL-6), interleukin-10 (IL-10); (5) growth factors, such as, for example, platelet-derived growth factor-AB (PDGF-AB), insulin-like growth factor I (IGF-I), transforming growth factor beta (TGF-β), epidermal growth factor (EGF), fibroblast growth factor (FGF) basic, granulocyte colony stimulating factor (G-CSF), granulocyte-macrophage colony-stimulating factor (GM-CSF), vascular endothelial growth factor (VEGF); (6) interferons, such as, for example, interferon-α (IFNα), interferon-γ (IFNγ); (7) C-reactive protein (CRP); and (8) macrophage inflammatory proteins (MIPs), such as, for example, macrophage inflammatory protein-1 alpha (MIP-1α), macrophage inflammatory protein-1 beta (MIP-1β) and macrophage inflammatory protein-2 (MIP2). In general, the protease compositions and methods of use of the present disclosure promote wound healing, prevent scarring, improve skin tone and stimulate the development of a smooth, healthy skin.

The present disclosure also provides protease compositions and methods of use that modulate the activity of at least one inflammatory or pro-inflammatory protein, thereby preventing or treating inflammation. The inflammation-related proteins can include, but are not limited to: (1) TNFα; (2) interleukins, such as, for example, IL-β. IL-6 and IL-10; (3) serum amyloid A; (4) fibrinogen; (5) interferons, such as, for example IFNα and IFNγ; (6) CRP; (7) MIPs, such as, for example, MIP-1α, MIP-1β and MIP2; and (8) MMPs, such as, for example, MMP-2, MMP-3 and MMP-9. Furthermore, since wound healing and inflammation generally go hand-in-hand, the wound-related proteins listed above play a role in inflammation, and thus, many wound-related proteins are also included in the category of inflammation-related proteins.

The term “wound” as used herein, refers to a tissue lesion or area of destruction caused by external factors or the presence of an underlying physiological disorder. The wounds may be localized or cover a large area of skin and tissue surface, and may either be open or have intact skin or tissues covering the area. Wounds or damaged tissue may be cutaneous in nature, but may also be found in other tissues throughout the body. The external factors that cause dermatologic wounds to essentially develop are commonly irradiation, mechanical, thermal or chemical trauma. As a consequence of their formation, tissue lesions lead to blood and fluid loss and decreased function, while disruption of the protective function of the skin can allow pathogens, foreign bodies and various toxins to enter the body.

According to the present disclosure, protease compositions having one or more proteases is useful for the treatment of wounds and skin conditions, such as, for example, inflammation. Administration of such a mixture modulates the activity of wound-related proteins, and diminishes the rate of tissue destruction, inflammation, edema, fever, pain, itching and hyperpermeability of endothelium in wounds. Hence, such a protease composition can provide an improvement in wound healing. Additionally, the administration of such a composition degrades inflammation-related proteins and diminishes the intensity of inflammation in skin or wounds. Hence, such a protease composition can improve wound healing processes by providing a faster rate of resolution to inflammation as well as decrease scarring.

Additionally, an embodiment of the present disclosure provides protease compositions and methods of use that are useful for the management of the environment in and around various cells, such as, for example, pre-cancerous and cancerous cells. These cells secrete enzymes, cytokines and growth factors in order to evade the immune system and to establish a blood supply. The protease compositions and methods of use of the present disclosure can be used to modulate the microenvironment of these cells (e.g., pre-cancerous and cancerous cells) in a subject, thereby promoting a normal environment and diminishing the ability of these cells to establish a permanent foothold at their location by thwarting their manipulative and subversive use of MMPs and certain cytokines and growth factors, such as, for example, FGF basic, VEGF, PDEGF, angiopoietin-2 (Ang2) and ephrin B2 (EFNB2). If the microenvironment returns to normal, the cellular environment (e.g., cellular environments having pre-cancerous and cancerous cells) can fall prey to the immune system and lack of nutrients, but without the adverse side effects of chemotherapy, thereby promoting healing and improved health.

Most protein modulation strategies involve preventing activity of the respective proteins with organic small molecules. These compounds are often toxic to the body and are not naturally occurring molecules. Use of natural polypeptides, such as, for example, proteases to modulate protein levels and activity provides a high degree of proteinase control without toxic side effects. Unlike small molecule inhibition strategies, the protease compositions and methods of use of the present disclosure can be used to degrade specific proteins, such as, for example, MMPs, while leaving growth factors and other beneficial polypeptides intact. The protease compositions of the present disclosure can be freely introduced onto the skin, into the wound environment or can be tethered to, or delivered by, an appropriate carrier or vehicle depending on the wound.

The protease compositions and methods of use of the present disclosure provide a high degree of control over the level of wound-related and inflammation-related protein activity for healing chronic wounds. For example, as some amount of MMP level is required during chronic wound healing, one of ordinary skill in the art may choose to only partially inhibit the activity of one or more MMPs. As such, by varying the type and concentrations of proteases applied, the degree of protein degradation, such as, for example, MMP degradation, and consequently inhibition, can be controlled.

One of ordinary skill in the art can choose an appropriate protease or combination of proteases to achieve the quality and quantity of modulation desired using available teachings in combination with the teachings provided herein. As used herein, the term “modulation” refers to the variation of the native activity or levels of a protein. Thus, the process of modulation can involve inhibition of a particular protein's activity via degradation or other means. Alternately, modulation of a protein's activity can take the form of an activation step, for example, the activation of a pro-enzyme to its active enzymatic form via degradation or other means. “Quality” of inhibition or activation refers to the type of protein targeted. For example, different MMPs can have somewhat different substrates and sites of activity. “Quantity”, as used herein, of inhibition or activation refers to the overall amount of inhibition or activation from all proteins that are targeted by the protease compositions. The type and quantity of protease or proteases used determines the level of inhibitory or activation modulatory effects on the target protein or proteins. One of ordinary skill in the art can readily make modifications to the protease compositions and methods of use provided by the present disclosure and observe the type and degree to which a given protein, such as, for example, an MMP is inhibited.

According to the various aspects of the present disclosure, a protease composition and methods of use that is useful for wound healing, reducing inflammation and promoting development of healthy skin are provided. As provided herein, the term “protease” is used synonymously with the term “proteinase” or “peptidase”. The protease compositions and methods of use provided by the present disclosure inhibit the activity of many types of MMPs, primarily, for example, by degradation of the MMPs. Moreover, the protease compositions and methods of use can be adjusted so that they inhibit a broad spectrum of metalloproteinases. Alternately, the protease compositions and methods of use can be modified so that only one or a few select metalloproteinases are inhibited. The protease compositions and methods of use of the present disclosure can inhibit the activity of many types of MMPs. The protease compositions and methods of use of the present disclosure can also prevent the activation of proenzyme MMPs, as well as inhibit the enzymatic activity of mature MMPs.

In certain embodiments of the present disclosure the protease compositions and methods of use can be changed so that certain proteins, including, but not limited to, MMPs, are activated. In certain types of activation, the pro-form of a protein is activated to form a mature form of the protein. Such an activation process provides an active protein that is capable of participating in the wound healing process. An example of this type of activation is the use of proteases to activate specific MMPs to modulate wound environments of wounds displaying keloids or exuberant granulation tissue formation. In these types of wounds or scars, excessive amounts of ECM, collagen and granulation tissue are deposited. The amount can be so great that the wound cannot close, or may form so much excessive tissue, it appears as a tumor protrudance. These types of wounds and scars are a result of a dysfunctional micro-environment in which too few MMPs are active, fibroblast secrete collagen unregulated or cytokine and growth factors are depressed (e.g., IFNγ) or expressed in excess (e.g., TNFα or IL-6). Application of an embodiment of the present disclosure, with or without surgical intervention, can modulate the micro-environment to promote a return to normal wound healing or normal scar remodeling.

The protease compositions and methods of use provided by the present disclosure can inhibit the activity of many types of proteins, primarily by degradation. As such, an embodiment of the present disclosure provides protease compositions and methods of use that are capable of broadly inhibiting a large number of different proteins. Another embodiment of the present disclosure provides protease compositions and methods of use that inhibit either a single protein or selected proteins. A further embodiment of the present disclosure provides protease compositions and methods of use that can activate one or more proteins. The activation of the protein occurs via cleavage of a dormant, or less-active form, which provides an active form of the protein. The protease compositions and methods of use of the present disclosure can modulate the activity of many types of proteins. The protease compositions and methods of use of the present disclosure can also prevent the activation of pro-forms of protein molecules, as well as inhibit the enzymatic activity of mature forms of protein molecules. Another embodiment of the present disclosure provides protease compositions and methods of use that inhibit one or more proteins and activates one or more different proteins. Additional embodiments of the present disclosure provide protease compositions and methods of use that elevate or eliminate itch in a wound or damaged tissue. According to an embodiment of the present disclosure, protease compositions and methods of use can selectively degrade certain proteins, such as, for example, MMPs or inflammation-related proteins at the site of the wound, while beneficial proteins, such as, for example, TIMP-1 and PDGF are spared from degradation, that is, certain proteins are resistant to degradation, while others undergo proteolytic degradation.

The proteolytic activity of a protease can be assessed by any procedure available to one of ordinary skill in the art. Many different assay procedures are available to determine whether or not a particular protease or mixture of proteases exhibit proteolytic activity. One such technique is an enzyme-linked immunosorbent assay (ELISA) assay.

According to the present disclosure, the protease composition can include one or more proteases. The protease compositions can include, for example, at least one hydrolase enzyme, such as, for example, aminopeptidase, aspartic endopeptidase, cysteine endopeptidase, cysteine-type carboxypeptidase, dipeptidase, dipeptidyl-peptidase, metallocarboxypeptidase, metalloendopeptidase, omega peptidase, peptidyl-dipeptidase, serine endopeptidase, serine-type carboxypeptidase, tripeptidyl-peptidase, threonine endopeptidase families, and combinations of the same and like.

Examples of proteases can include, but are not limited to, acrosin, actinidain, ananain, asclepain, aspergillopepsin I, bacterial leucyl aminopeptidase, brachyurin, bromelain, calpain, carboxypeptidase A, caricain, cathepsin, chymopapain, chymosin, chymotrypsin, complement subcomponent C1r, cytosol aminopeptidase, DD-transpeptidase, dipeptidyl peptidase, deuterolysin, elastase, enteropeptidase, ficain, fragilysin, glycyl endopeptidase, hypodermin, ingensin, kallikrein, kininase, L-peptidase, methionine aminopeptidase, papain, pepsin, peptidyl-glycinamidase, plasmin, proproteinase, semenogelase, streptogrisin, subtilisin, thrombin, and combinations of the same and like.

In some instances, the use of bacterial leucyl aminopeptidase can result in the release of an N-terminal amino acid, thus inactivating the certain target molecule functions. In an additional example, the use of a complement subcomponent C1r protease can selectively cleave the bond in complement subcomponent C1s to activate form of C1s, which then can activate C2 and C4. In a further example, the use of fragilysin can hydrolyzes a variety of bonds of extracellular matrix proteins.

In various embodiments, other conditions which may be treated or prevented by the protease compositions and methods of use of the present disclosure can include, but are not limited to, inflammatory diseases, autoimmune diseases and neurodegenerative diseases.

Inflammatory diseases which may be treated or prevented can include, for example, septic shock, septicemia and adult respiratory distress syndrome. Target autoimmune diseases include, for example, rheumatoid arthritis, systemic lupus erythematosus, scleroderma, chronic thyroiditis, Graves' disease, autoimmune gastritis, insulin-dependent diabetes mellitus, autoimmune hemolytic anemia, autoimmune neutropenia, thrombocytopenia, chronic active hepatitis, myasthenia gravis and multiple sclerosis. Neurodegenerative diseases can include, for example, amyotrophic lateral sclerosis, Alzheimer's disease, Parkinson's disease and primary lateral sclerosis. Additionally, other diseases associated with harmful, apoptosis are readily envisioned, such as those associated with ischemic injury, includes myocardial infarction, stroke and ischemic kidney disease for example. Moreover, the protease compositions and methods of use of the present disclosure can additionally be used to treat infectious diseases, especially those involved with microbial, parasitic and viral infections.

Furthermore, other inflammation inducing conditions may be treated to ameliorate symptoms associated with inflammation or to diminish the existing inflammation. Inflammation or irritation associated therewith may be from a variety of sources either physical or chemical as discussed above, and may include, without limitation, insect bites or stings, contact with a particular type plant (e.g., poison oak, etc.), radiation (e.g., ultraviolet radiation), non-infectious conjunctivitis, ophthalmic injuries, tonsillitis, hemorrhoids (acute), abrasions, ingrown finger or toenails (granulation), skin graft donor sites, vaginitis, dermatitis, psoriasis, herpes simplex (e.g., cold sores or aphthous ulcers), pruritus ani/cruri, chemical inflammation, cystic fibrosis and the combinations of the same and like. Moreover, such inflammation or other activities of the MMP family of proteases may lead to lack of elasticity or diminished skin appearance and texture or decreased tissue function. Accordingly, the protease compositions and methods of use set forth herein find utility not only in treating inflammatory diseases, but also for in treatment of the associated conditions and symptoms.

Inflammation is the result of extraneously or intrinsically induced damage to cells or tissue. Such damage may be induced by chemical or physical influences upon the skin or mucus membranes of humans and animals. Examples of physical influences include, but are not limited to, infarction, heat, cold, radiation and electrical shock, and examples of chemical influences include, but are not limited to, contact with acids, bases and allergens. Additionally, inflammation may be induced by microorganisms acting on the skin, as well as being the result of microorganisms invading the human or animal body.

In general, a variety of symptoms are associated with inflammation and can include, but are not limited to, one or more of pain, increased surface temperature, heat, redness, whelps, hives, edema, swelling, itching, pruritus, pain, and reduced or ceased function. The inflammatory responses that may be ameliorated may be on the skin or a mucus membrane of a human or animal, such as, for example, a mammal, and can include, but is not limited to, conditions such as inflammation around erupting wisdom teeth, following extraction of teeth, periodontal abscesses, prosthesis induced pressure sores on the mucosa, fungal infections, for treating exposed bone surface in alveolitis sicca dolorosa, which is a painful condition which may arise following extraction of teeth, chronic and acute inflammatory diseases including, but not limited to, pancreatitis, rheumatoid arthritis, osteoarthritis, asthma, inflammatory bowel disease and psoriasis.

Moreover, several morphological changes, including, but not limited to, a decreased moisture content of the stratum corneum, coupled with reduced eccrine and sebaceous gland output can decrease the presence of these components which protect the skin and allow for loss of collagen, the major skin protein. These morphological changes which result in a loss of integrity of the horny layer of the skin can be caused by a variety of conditions. Among such conditions are environmental (e.g., sun or wind exposure), trauma or wounds (e.g., cuts, burns or abrasions), exposure to chemicals (e.g., chemicals such as alkaline soaps, detergents, liquid solvents, oils or preservatives), and diseases (e.g., eczema, psoriasis or seborrheic dermatitis). Accordingly, protease compositions and methods of use that suppress the protease activity of the MMP family of proteases are useful in maintaining the skin.

Protease compositions and methods of use of the present disclosure can be used to heal wounds, and are particularly beneficial for chronic wound healing. Individual proteases, protease variants, polypeptide derivatives and mixtures thereof (e.g., those with different sequences) can be combined in a formulation to promote wound healing and to prevent or treat skin problems. Optimal healing and skin regeneration may require some matrix metalloproteinase activity, and hence, the protease compositions and methods of use of the present disclosure do not necessarily promote maximal inhibition of matrix metalloproteinases. Rather, in some instances, the activity of the polypeptide inhibitor formulation is varied as needed to optimize healing and promote healthy skin development. For example, lesser or greater levels of inhibition can be achieved by varying the type, content and amount of inhibitor polypeptides so that healing and healthy skin development is promoted. Depending on the wound etiology, the patient immune system and the tissue trauma, various formulations of the protease compositions and methods of use can be developed in order to provide an optimal protein and enzyme activation and inactivation ratios specific for the disease.

To promote healthy skin development or treat wounds, protease compositions and methods of use of the present disclosure can be introduced onto the skin or tissues, or into wounds in any manner chosen by one of ordinary skill in the art. For example, proteases can be formulated into a therapeutic composition containing a therapeutically effective amount of one or more proteases and, for example, can also contain a pharmaceutical carrier. Such protease compositions can be introduced onto skin or into the wound as, for example, a cream, spray, foam, gel, solution or in any other form or formulation. In some embodiment, the protease compositions and methods of use of the present disclosure can be formulated into a skin covering or dressing containing a therapeutically effective amount of one or more proteases impregnated into, covalently attached to a covering or dressing material, or otherwise associated with a covering or dressing material. In some embodiments, the skin covering or dressing permits release of the protease composition. Release of the protease composition can be conducted in an uncontrolled or a controlled manner. Hence, the skin coverings or wound dressings of the present disclosure can provide a slow or timed release of the protease composition into a wound. Skin coverings and dressing materials can be any material used known to those of ordinary skill in the art, and can include, but are not limited to, bandages, gauze, sterile wrappings, hydrogels, hydrocolloids and combinations of the same or similar materials.

In general, a therapeutically effective amount of a protease composition of the present disclosure is an amount of one or more proteases that modulate the target protein activity or levels, such as, for example, an MMP, to a degree needed to promote healthy tissue development or wound healing. For example, when present in a therapeutic or pharmaceutical composition, the concentration of the one or more proteases can be in the range of about 0.001% to about 35% by weight of the composition. Additionally, the one or more proteases can form about 0.5% to about 20% by weight of the composition. Alternately, the one or more proteases can form about 1.0% to about 10% by weight of the composition. The therapeutically effective amount of protease necessarily varies with the route of administration. Furthermore, the amount of the protease required for healthy skin development or wound treatment will vary not only with the route of administration, but also the nature of the condition being treated and the age and condition of the patient, and will be ultimately at the discretion of the attendant physician or clinician. The dosage and methods of administration can also vary depending upon the location of the skin or tissue to be treated, or based, at least in part, upon the severity of the wound.

The protease compositions of the present disclosure can be formulated as pharmaceutical compositions and administered to a mammalian host, such as, for example, a human patient in a variety of dosage forms adapted to the chosen route of administration, for example, orally or parenterally, by intravenous, intramuscular, inhalation, topical or subcutaneous routes. Thus, the protease compositions may be systemically administered, for example, intravenously or intraperitoneally by infusion or injection. Solutions of the protease compositions can be prepared in water, optionally mixed with a nontoxic surfactant. Dispersions can also be prepared in glycerol, liquid polyethylene glycols, triacetin and mixtures thereof, and in oils. In general, and under ordinary conditions of storage and use, these preparations can additionally contain a preservative to prevent the growth of microorganisms.

Topical products can be formulated with a variety of carriers and solvents to enhance the stability, bioavailability, and delivery of active ingredients to the skin. Some common types of carriers and solvents used in topical formulations include:
Water-based carriers: Water is the most commonly used carrier in topical products. It is usually combined with other ingredients to form emulsions or gels. Examples of water-based carriers include distilled water, purified water, and deionized water.
Oil-based carriers: Oil-based carriers are used to solubilize and deliver lipophilic (fat-soluble) active ingredients. Examples of oil-based carriers include mineral oil, vegetable oils, and silicone oils.
Alcohols: Alcohols are used as solvents and penetration enhancers in topical formulations. Examples of alcohols used in topical products include ethanol, isopropyl alcohol, and butanol.
Glycols: Glycols are used as humectants, solvents, and penetration enhancers. Examples of glycols used in topical formulations include propylene glycol, butylene glycol, and hexylene glycol.
Surfactants: Surfactants are used to emulsify oil and water-based carriers to form stable emulsions. Examples of surfactants used in topical formulations include polysorbates, sodium lauryl sulfate, and cetyl alcohol.
Polymers: Polymers are used to thicken topical formulations and improve their stability. Examples of polymers used in topical products include carbomers, cellulose derivatives, and polyvinylpyrrolidone.

To form emulsions, lotions, creams foams or gels, the different carriers and solvents and other ingredients are combined in specific ratios and are mixed under controlled conditions, such as temperature and mixing speed. The type and concentration of the carriers, ingredients and solvents used in the formulation will determine the format of the final resulting formulation which includes any type of emulsion, cream, lotion, foam, or gel.

Commonly used ingredients in protease containing formulations include thickeners, humectants, preservatives, and other ingredients, which are added to provide specific properties or functions.

Some examples of thickeners include Xanthan gum, a natural thickener that is commonly used in concentrations of 0.1% to 5%; Carbomers, which are synthetic thickeners that is commonly used in concentrations of 0.1% to 10%; Hydroxyethyl cellulose, another thickener that is commonly used in concentrations of 0.1% to 15%.

Some examples of humectants include Glycerin, a natural humectant that is commonly used in concentrations of 1% to 45%; Propylene glycol, a synthetic humectant that is commonly used in concentrations of 1% to 5%; Sodium hyaluronate, a natural humectant that is commonly used in concentrations of 0.1% to 1%.

Some examples of preservatives include Phenoxyethanol, a preservative that is commonly used in concentrations of 0.5% to 1%; Ethylhexylglycerin, a preservative that is commonly used in concentrations of 0.5% to 1%; Potassium sorbate, a preservative that is commonly used in concentrations of 0.1% to 0.5%.

Vitamins and antioxidants can be added to protease formulations to provide additional skin benefits. The concentration will depend on the specific ingredient and its recommended usage rate. Extracts and essential oils can be added to all formulations to improve the user experience. The concentration will depend on the desired strength of the fragrance and any restrictions on use.
Emulsions: Oil in water (O/W) and water in oil (W/O) emulsions are two common types of emulsions used in topicals. An O/W emulsion consists of small droplets of oil dispersed within a continuous phase of water. This type of emulsion is commonly used in products that are intended to feel lightweight and non-greasy, such as lotions and serums. A W/O emulsion consists of small droplets of water dispersed within a continuous phase of oil. This type of emulsion can be commonly used in products that are intended to provide more moisture and a heavier, more emollient feel, such as creams and balms. Gels are formed when a polymer is added to a solvent, such as water or alcohol, and the mixture is allowed to swell and form a gel-like consistency. The type of emulsion or gel formed will affect the texture, appearance, and stability of the final product.

To create an O/W emulsion, an emulsifier is typically used to stabilize the oil droplets and prevent them from coalescing and separating from the water phase. The emulsifier molecules have a hydrophilic (water-loving) end and a hydrophobic (oil-loving) end, which allows them to form a layer around the oil droplets and keep them suspended in the water phase. The concentration of the emulsifier used can affect the stability and texture of the emulsion. Some common emulsifiers used in O/W emulsions include Polysorbate 20, a nonionic surfactant that is used to emulsify oils in water. It is commonly used in concentrations of 1% to 10%; Cetearyl alcohol, an emollient and emulsifying wax that is commonly used in concentrations of 2% to 10%; Glyceryl stearate, an emulsifying agent that is derived from vegetable oils and other sources can be commonly used in concentrations of 1% to 10%.

To create a W/O emulsion, an emulsifier is typically used to stabilize the water droplets and prevent them from coalescing and separating from the oil phase. The emulsifier molecules have a hydrophilic (water-loving) end and a hydrophobic (oil-loving) end, which allows them to form a layer around the water droplets and keep them suspended in the oil phase. The concentration of the emulsifier used can affect the stability and texture of the emulsion.

Some common emulsifiers used in W/O emulsions include Beeswax, a natural emulsifier that is commonly used in concentrations of 1% to 10%; Sorbitan oleate, a nonionic surfactant that is used to emulsify water in oil and can be used in concentrations of 1% to 10%; Hydrogenated castor oil, an emulsifying agent that can be used in concentrations of 0.5% to 15%.

Some examples of other ingredients used in an emulsion are silicone oil, a conditioning agent that is commonly used in concentrations of 1% to 5%; Allantoin, a soothing agent that is commonly used in concentrations of 0.1% to 50%.
Gel: Gel formulations can be made from a variety of ingredients depending on their intended use and the properties desired in the final product. Examples of ingredients commonly used in gel formulations are Gelling agents which include Carbomers, Xanthan gum, Hydroxyethylcellulose. Humectants are also used in gels and the commonly used ones are Glycerin which can be used from 2% to 35%, Propylene glycol which can be used from 1% to 25%, Butylene glycol which can be used from 1% to 10%.
Foams: Foam formulations can be created from a variety of ingredients that can include thickeners, humectants preservatives and fragrances, depending on their intended use and the properties desired in the final product. Some examples of ingredients commonly used in foam formulations are Surfactants which include Sodium lauryl sulfate and Sodium laureth sulfate, anionic surfactants that are typically used at concentrations of 2% to 30%. Cocamidopropyl betaine, an amphoteric surfactant typically used at concentrations of 2% to 20%.
Lotions: Lotions and creams are two types of emulsion-based formulations that are designed to be applied to the skin for a variety of purposes, such as moisturizing, nourishing, and protecting the skin. The specific ingredients and their concentrations in these formulations can vary depending on the intended use, the desired texture, and the properties of the ingredients themselves. Examples of ingredients commonly used in lotion and cream formulations include emollients among other ingredients. Examples of emollients include mineral oil, typically used at concentrations of 1% to 20%; Petrolatum, which can be used at concentrations of 1% to 60%; Dimethicone, a silicone-based emollient that can be used from 1% to 15%.

It is important to note that the concentration ranges of these ingredients can vary depending on the specific product and its intended use. Additionally, the use of some of these ingredients, such as preservatives, is subject to regulations and guidelines set by regulatory agencies like the FDA, and their use in products must comply with these regulations. The concentration of each ingredient used should be determined, taking into consideration the intended use, the product's stability, and any potential for skin irritation or other adverse effects.

The pharmaceutical dosage forms suitable for injection or infusion, or topical application can include sterile aqueous solutions or dispersions, or sterile powders having active ingredients that are adapted for the extemporaneous preparation of sterile injectable or infusible solutions, or dispersions, optionally encapsulated in liposomes. In general, the ultimate dosage form should be sterile, fluid and stable under the conditions of manufacture and storage. The liquid carrier or vehicle can be a solvent or liquid dispersion medium including, for example, water, ethanol, a polyol (e.g., glycerol, propylene glycol, liquid polyethylene glycols and combinations of the same and like), vegetable oils, nontoxic glyceryl esters and suitable mixtures thereof. The proper fluidity can be maintained, for example, by the formation of liposomes or by the maintenance of the required particle size in the case of dispersions, or by the use of surfactants. The prevention of the actions of microorganisms can be brought about by various antibacterial and antifungal agents, for example, parabens, chlorobutanol, phenol, sorbic acid, thimerosal and combinations of the same and like. In some cases, one of ordinary skill in the art can choose to include isotonic agents, for example, sugars, buffers or sodium chloride. Prolonged absorption of the injectable compositions can be brought about by the use of agents delaying absorption, for example, aluminum monostearate and gelatin.

Sterile injectable solutions can be prepared by incorporating the protease compositions or a protease conjugate in the required amount in the appropriate solvent with various of the other ingredients enumerated above, as required, followed by sterilization. In the case of sterile powders for the preparation of sterile injectable solutions, methods of preparation can include, without limitation, vacuum drying and the freeze-drying techniques, which yield a powder of the active ingredient plus any additional desired ingredient present in the previously sterile solutions.

In some instances, the protease compositions can also be administered orally, in combination with a pharmaceutically acceptable vehicle, such as, for example, an inert diluent or an assimilable edible carrier. The protease compositions can be enclosed in hard- or soft-shell gelatin capsules, can be compressed into tablets or can be incorporated directly with the food of a patient's diet. For oral therapeutic administration methods, the protease compositions can be combined with one or more excipients and used in the form of ingestible tablets, buccal tablets, troches, capsules, elixirs, suspensions, syrups, wafers and combinations of the same and like. Such compositions and preparations can contain at least 0.1% by weight of active compounds. The percentage of the compositions and preparations may, of course, be varied and may conveniently be between about 2% to about 60% of the weight of a given unit dosage form. The amount of active compound in such therapeutically useful compositions is such that an effective dosage level will be obtained.

In some instances, tablets, troches, pills, capsules, and combinations of the same and like can also contain the one or more of the following, such as, binders (e.g., gum tragacanth, acacia, corn starch or gelatin), excipients (e.g., dicalcium phosphate), a disintegrating agent (e.g., corn starch, potato starch, alginic acid and combinations of the same and like), a lubricant (e.g., magnesium stearate), a sweetening agent (e.g., sucrose, fructose, lactose or aspartame) or a flavoring agent (e.g., peppermint, oil of wintergreen or fruit flavoring such as cherry or orange). When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For example, tablets, pills or capsules may be coated with gelatin, wax, shellac, sugar or combinations of the same and like. A syrup or elixir may contain the active compound, sucrose or fructose as a sweetening agent, methyl and propylparabens as preservatives, a dye and flavoring such as cherry or orange flavor. However, any material used in preparing any unit dosage form should be pharmaceutically acceptable and substantially nontoxic in the amounts employed. In addition, the polypeptide inhibitor may be incorporated into sustained-release preparations and devices.

In general, useful solid carriers can include, without limitation, finely divided solids, such as, for example, talc, clay, microcrystalline cellulose, silica, alumina or combinations of the same and like. Additionally, useful liquid carriers can include, but are not limited to, water, alcohols or glycols, or water-alcohol/glycol blends, in which the protease compounds can be dissolved or dispersed at effective levels, optionally with the aid of nontoxic surfactants. Furthermore, adjuvants, such as, for example, fragrances and additional antimicrobial agents can be added to optimize the properties for any given use. Additionally, thickeners, such as, for example, synthetic polymers, fatty acids, fatty acid salts and esters, fatty alcohols, modified celluloses or modified mineral materials can also be employed with liquid carriers to form spreadable pastes, gels, ointments, soaps and combinations of the same and like, for application directly to the skin of the user.

In general, the protease compositions of the present disclosure can be administered topically for wound treatment and for promoting healthy skin development. The active polypeptides can be administered topically by any means either directly or indirectly to the selected tissue as sprays, foams, powders, creams, jellies, pastes, suppositories or solutions. The term “pastes” as used herein should be taken to include, for example, creams and other viscous spreadable compositions such as are often applied directly to the skin or spread onto a bandage or dressing. The protease compositions of the present disclosure can be covalently attached, stably adsorbed or otherwise applied to a skin covering or wound dressing material. To facilitate healing after surgery, the active proteases of the present disclosure can be applied directly to target tissue, prosthetic devices or implantable sustained released devices. The protease compositions can be administered by aerosol, as a foam or as a mist, or gel or solution, with or without other agents, directly onto the skin or wound.

The protease compositions can be administered in a formulation that can include, for example, an emulsion of the protease in a wax, oil, an emulsifier, water or a substantially water-insoluble material that forms a gel in the presence of water. The formulations can provide the desirable properties of an emulsion such that it is spreadable and has the creamy consistency of an emulsion, yet that does not break down when subjected to normal sterilization procedures, for example, steam sterilization, because the gel stabilizes the emulsion. These also exhibits better water retention properties than a conventional gel because water is held both in the emulsion and in the gel.

The protease compositions of the present disclosure can also contain a humectant to reduce the partial vapor pressure of the water in the cream or lotion to reduce the rate at which the cream or lotion dries out. Suitable humectants are generally miscible with water to a large extent and are generally suitable for application to the skin. Polyols are generally suitable for this purpose, and suitable polyols can include, without limitation, mono propylene glycol or glycerin (glycerol). The polyol can be present in proportions of about 20% to about 50% (by weight) of the total protease composition. Alternatively, the range can be about 30 to about 40%. This relatively high proportion of polyol can ensure that if a paste should dry out to any degree, the resulting paste remains soft and flexible as the glycerin can act as a plasticizer for the polymer. When a paste is applied on a bandage, for example, it may therefore still be removed easily from the skin when the paste has lost water without the need to cut the bandage off. The polyols also have the advantage of functioning to prevent the proliferation of bacteria in the paste when it is in contact with the skin or wound, particularly infected wounds.

In some instances, the protease compositions can include other ingredients, such as, for example, antibacterial agents, antifungal agents, anti-inflammatory agents and combinations of the same and like. Additionally, other ingredients can also be found suitable for incorporation into the formulation, such as, for example, vitamins and herbal agents.

Proteases can be used in topical formulations to improve the bioavailability and efficacy of antibacterial agents, antifungal agents, anti-inflammatory agents, vitamins, herbs and combinations of the same and like.

Some examples of antibacterial agents that are commonly found in formulations with proteases include benzoyl peroxide, Clindamycin, Mupirocin among others. Benzoyl peroxide is an antibacterial agent that is commonly used in acne treatments. It works by releasing oxygen into the pores, which helps to kill the bacteria that cause acne. Benzoyl peroxide is often formulated with proteases to improve its efficacy and reduce skin irritation. Clindamycin is an antibiotic that is used to treat bacterial infections on the skin, such as acne and folliculitis. Clindamycin can be formulated with proteases to improve its penetration into the skin and increase its bioavailability. Mupirocin is an antibiotic that is used to treat bacterial skin infections, such as impetigo and folliculitis. Mupirocin can be formulated with proteases to improve its efficacy and reduce the risk of bacterial resistance.

The concentration ranges of antibacterial agents in formulations with proteases can vary depending on the specific product and its intended use. For example, benzoyl peroxide is often formulated at concentrations of 2.5% to 10%, while clindamycin can be formulated at concentrations of 1% to 2%. Mupirocin is typically formulated at concentrations of 2% to 5%.

Some examples of antifungal agents that are commonly found in formulations with proteases include Clotrimazole, Terbinafine and Ketoconazole among others. Clotrimazole is an antifungal agent that is used to treat fungal infections on the skin, such as ringworm and jock itch. Clotrimazole can be formulated with proteases to improve its penetration into the skin and increase its bioavailability. Terbinafine is an antifungal agent that is used to treat fungal infections on the skin, such as athlete's foot and ringworm. Terbinafine can be formulated with proteases to improve its efficacy and reduce the risk of fungal resistance. Ketoconazole is an antifungal agent that is used to treat a variety of fungal infections, including skin infections. Ketoconazole can be formulated with proteases to improve its bioavailability and reduce skin irritation.

The concentration ranges of antifungal agents in formulations with proteases can vary depending on the specific product and its intended use. For example, clotrimazole is often formulated at concentrations of 1% to 2%, while terbinafine can be formulated at concentrations of 1% to 2%. Ketoconazole is typically formulated at concentrations of 2% to 4%.

Some examples of anti-inflammatory agents that are commonly found in formulations with proteases include Hydrocortisone, Diclofenac and Salicylic acid among others. Hydrocortisone is a corticosteroid that is used to reduce inflammation and itching on the skin. Hydrocortisone can be formulated with proteases to improve its penetration into the skin and increase its bioavailability. Diclofenac is a nonsteroidal anti-inflammatory drug (NSAID) that is used to reduce inflammation and pain on the skin. Diclofenac can be formulated with proteases to improve its efficacy and reduce the risk of side effects associated with oral NSAIDs. Salicylic acid is a beta-hydroxy acid that is used to exfoliate the skin and reduce inflammation. Salicylic acid can be formulated with proteases to improve its penetration into the skin and increase its bioavailability.

The concentration ranges of anti-inflammatory agents in formulations with proteases can vary depending on the specific product and its intended use. For example, hydrocortisone is often formulated at concentrations of 0.5% to 2%, while diclofenac can be formulated at concentrations of 1% to 3%. Salicylic acid is typically formulated at concentrations of 0.2% to 2%.

Some examples of vitamins that are commonly found in formulations with proteases include Vitamin A, Vitamin C and Vitamin E among others. Vitamin A is a retinoid that helps improve skin texture and reduce the appearance of fine lines and wrinkles. Vitamin A can be formulated with proteases to improve its penetration into the skin and increase its bioavailability. Vitamin C is an antioxidant that is essential for collagen synthesis and skin health. Vitamin E is an antioxidant that helps protect the skin from free radical damage and promotes healing. Vitamin E can be formulated with proteases to improve its efficacy and reduce skin irritation.

The concentration ranges of vitamins in formulations with proteases can vary depending on the specific product and its intended use. For example, vitamin C is often formulated at concentrations of 1% to 20%, while vitamin E can be formulated at concentrations of 0.1% to 2%. Vitamin A is typically formulated at concentrations of 0.025% to 1%.

Some examples of herbs that are commonly found in formulations with proteases include Aloe vera, Chamomile, Green tea. Aloe vera is a succulent plant that is commonly used in skin care products for its anti-inflammatory and moisturizing properties. Green tea is a plant that is rich in antioxidants and has been shown to have anti-inflammatory and anti-aging properties. Aloe vera and Green Tea can be formulated with proteases to improve its penetration into the skin and increase its bioavailability. Chamomile is a flower that is known for its soothing and anti-inflammatory properties. Chamomile can be formulated with proteases to improve its efficacy and reduce skin irritation.

The concentration ranges of herbs in formulations with proteases can vary depending on the specific product and its intended use. For example, aloe vera is often formulated at concentrations of 1% to 30%, while chamomile can be formulated at concentrations of 0.1% to 5%. Green tea is typically formulated at concentrations of 0.5% to 5%.

The concentration of these agents can be determined based on the specific product's intended use, as well as the potential for skin irritation and other adverse effects.

The concentration of proteases in these formulations can also vary, the concentration of the one or more proteases can be in the range of about 0. 0.000000001% to about 35% by weight of the composition. Additionally, the one or more proteases can form about 0.5% to about 20% by weight of the composition. Alternately, the one or more proteases can form about 1.0% to about 10% by weight of the composition.

An example of a wax for the emulsion is glyceryl monostearate, or a combination of glyceryl monostearate and polyethylene glycol-100 (PEG-100) stearate. This combination provides both a wax and an emulsifier (PEG-100 stearate) that is compatible with the wax for forming an emulsion in water. A second emulsifier can also be included in the formulation to increase the stability of the emulsion, for example, a polyethylene glycol-20 (PEG-20) stearate. In some instances, the total concentration of emulsifier in the cream can be in the range of from about 3% to about 15%. In cases where two emulsifiers are used, one may be present in a greater concentration than the other.

In general, the water-insoluble material forms a gel with the water of the formulation. The material is therefore hydrophilic, but does not dissolve in water to any great extent. The material can include a polymeric material, such as, for example, a water-absorbing non-water-soluble polymer. However, non-polymeric materials that form gels with water and that are stable at elevated temperatures could also be used, for example, clays, such as, but not limited to kaolin or bentonite. Some polymers used in the protease compositions of the present disclosure can be super-absorbent polymers that include hydrophilic cellulose derivatives that have been partially cross-linked to form a three-dimensional structure. Suitable cross-linked cellulose derivatives can include those of the hydroxy lower alkyl celluloses, where the alkyl group contains from about 1 to about 6 carbon atoms, for example, hydroxyethyl cellulose or hydroxypropyl cellulose, or carboxy-celluloses, for example, carboxymethyl hydroxyethyl cellulose or carboxy methylcellulose. An example of a polymer that may be used in the protease compositions of the present disclosure is a partially cross-linked sodium carboxy methylcellulose polymer. This particular polymer is a superabsorbent polymer in that it can absorb at least ten times its own weight of water. The cross-linked structure of the polymer prevents it from dissolving in water, but water is easily absorbed into, and held within the three-dimensional structure of the polymer to form a gel. Water is lost less rapidly from such a gel than from a solution and this can be advantageous in slowing or preventing the drying out of the cream formulations. The polymer content of the formulation is normally less than about 10%, and the polymer content can range from about 0.5% to about 5.0% by weight, or from about 1.0% to about 2% by weight.

In various instances, the protease compositions can be sterilized and components of the protease compositions can be selected, by varying the polymer content, to provide the desired flow properties of the protease product or compositions. That is, if the product is to be sterilized, then the formulation can be chosen to give the product a relatively high viscosity or elasticity before sterilization. If certain components of the protease compositions are not to be sterilized, the formulation can be sterilized before addition of those components, or each component can be sterilized separately. The protease compositions can then be made by mixing each of the sterilized ingredients under sterile conditions. When components are separately sterilized and then mixed together, polymer content can be adjusted to provide for the desired flow properties of the finished product or composition. The emulsion content can determine the handling properties and feel of the formulation, and higher emulsion content can lead to increased ability to spread and creaminess.

Sterilization by irradiation, as known by those of ordinary skill in the art does not lead to a decrease in activity of the proteases within the compositions. The protease compositions can be packaged into tubes, tubs or other suitable forms of containers for storage, or may be spread onto a substrate and then subsequently packaged. Suitable substrates can include, without limitations, dressings, including film dressings, bandages and combinations of the same and like. Because of their diverse applicability, the protease compositions of the present disclosure are also suitable for use as medicines, cosmetics, prescription drugs and over-the-counter (OTC) medications.

In various embodiments, the protease compositions of the present disclosure can contain one or more proteases in a solution at a concentration of approximately 10⁻⁸ to 2% (w/v). In some embodiments, the protease compositions of the present disclosure can contain one or more proteases in a solution at a concentration of approximately 10⁻⁸ to 1% (w/v).

For example, in some embodiments, the protease compositions of the present disclosure can contain one or more proteases in solution at a concentration of approximately 0.001 μg/ml to 0.01 μg/ml. In some embodiments, the protease compositions of the present disclosure can contain one or more proteases in solution at a concentration of approximately 0.01 μg/ml to 1.0 μg/ml. In some embodiments, the protease compositions of the present disclosure can contain one or more proteases in solution at a concentration of approximately 1.0 μg/ml to 10.0 μg/ml.

In some embodiments, the protease compositions of the present disclosure can contain one or more proteases in solution at a concentration of approximately 0.001 μg/ml. In some embodiments, the protease compositions of the present disclosure can contain one or more proteases in solution at a concentration of approximately 0.01 μg/ml. In some embodiments, the protease compositions of the present disclosure can contain one or more proteases in solution at a concentration of approximately 0.1 μg/ml. In some embodiments, the protease compositions of the present disclosure can contain one or more proteases in solution at a concentration of approximately 10.0 μg/ml.

In some embodiments, the protease compositions of the present disclosure can contain one or more proteases in solution at a concentration of approximately 0.001 mg/ml to 0.01 mg/ml. In some embodiments, the protease compositions of the present disclosure can contain one or more proteases in solution at a concentration of approximately 0.01 mg/ml to 0.1 mg/ml. In some embodiments, the protease compositions of the present disclosure can contain one or more proteases in solution at a concentration of approximately 0.1 mg/ml to 1.0 mg/ml.

In some embodiments, the protease compositions of the present disclosure can contain one or more proteases in solution at a concentration of approximately 0.001 mg/ml. In some embodiments, the protease compositions of the present disclosure can contain one or more proteases in solution at a concentration of approximately 0.01 mg/ml. In some embodiments, the protease compositions of the present disclosure can contain one or more proteases in solution at a concentration of approximately 0.1 mg/ml. In some embodiments, the protease compositions of the present disclosure can contain one or more proteases in solution at a concentration of approximately 1.0 mg/ml.

In some embodiments, the protease compositions of the present disclosure can contain one or more proteases in solution at a concentration of approximately 0.025 mg/ml to 0.25 mg/ml. In some embodiments, the protease compositions of the present disclosure can contain one or more proteases in solution at a concentration of approximately 0.25 mg/ml to 2.5 mg/ml. In some embodiments, the protease compositions of the present disclosure can contain one or more proteases in solution at a concentration of approximately 2.5 mg/ml to 10.0 mg/ml.

In some embodiments, the protease compositions of the present disclosure can contain one or more proteases in solution at a concentration of approximately 0.025 mg/ml. In some embodiments, the protease compositions of the present disclosure can contain one or more proteases in solution at a concentration of approximately 0.25 mg/ml. In some embodiments, the protease compositions of the present disclosure can contain one or more proteases in solution at a concentration of approximately 2.5 mg/ml. In some embodiments, the protease compositions of the present disclosure can contain one or more proteases in solution at a concentration of approximately 10.0 mg/ml.

In some embodiments, the protease compositions of the present disclosure can contain one or more proteases in solution at a concentration of approximately 0.5 mg/ml to 5.0 mg/ml. In some embodiments, the protease compositions of the present disclosure can contain one or more proteases in solution at a concentration of approximately 5.0 mg/ml to 10.0 mg/ml. In some embodiments, the protease compositions of the present disclosure can contain one or more proteases in solution at a concentration of approximately 10.0 mg/ml to 20.0 mg/ml.

In some embodiments, the protease compositions of the present disclosure can contain one or more proteases in solution at a concentration of approximately 0.5 mg/ml. In some embodiments, the protease compositions of the present disclosure can contain one or more proteases in solution at a concentration of approximately 5.0 mg/ml. In some embodiments, the protease compositions of the present disclosure can contain one or more proteases in solution at a concentration of approximately 10.0 mg/ml. In some embodiments, the protease compositions of the present disclosure can contain one or more proteases in solution at a concentration of approximately 20 mg/ml.

As described above, in some embodiments, the one or more proteases can include, but are not limited to, acrosin, actinidain, ananain, asclepain, aspergillopepsin I, bacterial leucyl aminopeptidase, brachyurin, bromelain, calpain, carboxypeptidase A, caricain, cathepsin, chymopapain, chymosin, chymotrypsin, complement subcomponent C1r, cytosol aminopeptidase, DD-transpeptidase, dipeptidyl peptidase, deuterolysin, elastase, enteropeptidase, ficain, fragilysin, glycyl endopeptidase, hypodermin, ingensin, kallikrein, kininase, L-peptidase, methionine aminopeptidase, papain, pepsin, peptidyl-glycinamidase, plasmin, proproteinase, semenogelase, streptogrisin, subtilisin, thrombin, and combinations of the same and like. In some embodiments, the one or more proteases can be a protease composition including a first protease. In some embodiments, the one or more proteases can be a protease composition including a combination of a first and second protease. In some embodiments, the one or more proteases can be a protease composition including a combination of a first, second and third protease. In some embodiments, the one or more proteases can be a combination of any number of different proteases.

In view of the aforementioned protease compositions, the present disclosure additionally pertains to treatment of wounds. In some embodiments, the wounds can include acute and chronic wounds. In some embodiments, the wounds can be the result of surgical incisions, burns, lacerations, ulcers, abrasions, and combinations of the same and like. Additionally, the wounds can be a tissue lesion or area of destruction caused by external factors or the presence of an underlying physiological disorder. Furthermore, the wounds can be localized or cover a large area of skin and tissue surface, and can either be open or have intact skin or tissues covering the area. Moreover, wounds or damaged tissue may be cutaneous in nature, but may also be found in other tissues throughout the body.

In some embodiments, the methods of treating a wound according to aspects of the present disclosure can include one or more stages of treatment. For example, in some embodiments, the stages can include a first stage (e.g., hemostasis phase), a second stage (e.g., inflammatory phase), a third stage (e.g., proliferative phase) and a fourth stage (e.g., maturation phase or remodeling stage).

Wound healing stages occur as a series of overlapping phases, i.e., hemostasis, inflammation, proliferation, and remodeling, with each phase involving a coordinated effort between a variety of cell types Neutrophils and macrophages are the main inflammatory cells that regulate these activities, maintaining a delicate balance between the proinflammatory and anti-inflammatory programs. While suppressed or short-lived inflammation might result in infection, excessive or chronic inflammation can cause tissue damage.

Hemostasis, the first stage of wound healing, begins as soon as tissue injury occurs in order to stop the blood flow at the injured spot. Endothelial cells form a single cell layer that lines blood vessels, maintaining a nonadherent surface so that, in the presence of normal blood, circulating platelets do not aggregate or stick to the vascular wall. When there is an injury, platelets are activated by collagen in the exposed subendothelial matrix, causing them to attach to the wound site and aid in the creation of a platelet plug. Thrombin, a vital enzyme in the coagulation cascade, engages platelet receptors to change circulating soluble fibrinogen fibers at the same time to produce stable clots at the location by polymerizing with platelets and turning into insoluble fibrin fibers. The clot works as a reservoir of cytokines, chemokines, and growth factors to facilitate the subsequent wound-healing stages and serves as a temporary matrix for recruited cells to infiltrate the wound.

The clot's platelet-derived growth factor (PDGF), which is generated by the platelets, attracts polymorphonuclear neutrophils (PMNs) to the wound site, starting the inflammatory phase of wound healing. The most prevalent inflammatory cells in circulation, neutrophils are ready to start an inflammatory response to counter the influx of invasive microorganisms. Within minutes after the first injury, there is a significant influx of neutrophils at the wound site, where they release a variety of antimicrobial agents that kill contaminating bacteria and other organisms.

After a few days, neutrophils continue to infiltrate; however, their numbers start to decline after 48 hours, at which point they are phagocytosed by macrophages made of monocytes drawn from the peripheral circulation. These macrophages debride the lesion in addition to eliminating exhausted neutrophils, removing debris and nonviable tissue. Macrophages produced from monocytes are essential to the healing of wounds. Macrophages exhibit a “classical” inflammatory response early on in the inflammatory phase.

Proinflammatory cytokines such Interleukin 12 (IL-12), IL-1, IL-6, tumor necrosis factor (TNF), and inducible nitrous oxide synthase are expressed during the activation state (also known as M1-like) (iNOS). These proinflammatory macrophages are in charge of clearing the wound of neutrophils and debris as well as any lingering microbial infections by inducing apoptosis in neutrophils.

The environment around the wound changes from being pro-inflammatory to being one that encourages tissue repair and remodeling as the inflammatory phase of the wound develops. Macrophages actively regulate this transition as they begin to differentiate into an “alternatively activated,” or M2-like, state secreting anti-inflammatory cytokines such as IL-4, IL-10, IL-13, and transforming growth factor β (TGFβ) as well as vascular endothelial growth factor (VEGF) and PDGF. M2-like macrophages begin to dominate the wound and levels of the more proinflammatory M1-like macrophages are reduced. However, it should be noted that, in vivo, distinct phenotypes such as M1 and M2 macrophages are rarely seen but exist along a spectrum between pro- and anti-inflammatory phenotypes. It is unclear what causes the change in macrophage phenotype. Nonetheless, the passage from proinflammatory macrophages to anti-inflammatory macrophages in the wound is necessary for advancement to the subsequent proliferative phase.

In order to attract and activate fibroblasts and keratinocytes and initiate the proliferative phase, macrophages produce chemokines and growth factors as they change to a reparative phenotype. By secreting proteinases and matrix metalloproteases (MMPs), fibroblasts destroy the temporary matrix created during hemostasis and replace it with granulation tissue via deposition of collagen and other extracellular matrix (ECM) proteins. Later on in the proliferative phase, keratinocytes move across the granulation tissue's surface and take the epithelium's place.

The final phase of wound healing, the remodeling phase, begins once granulation tissue is present and, thus, overlaps with the proliferation phase. During this phase, the granulation tissue is remodeled to more closely resemble normal skin. Fibroblasts differentiate into contractile myofibroblasts and replace the collagen type III that makes up the majority of the granulation tissue with the stronger, more rigid collagen type I.

Further, fibroblasts secrete elastin, which is responsible for elasticity of the skin. The final product of these concerted processes is a scar that is mechanically functional. However, hair follicles, sweat glands, and nerves do not recover and the tissue retains only 50%-70% of its original strength

In some embodiments, fewer or more stages of treatment can be pre-formed depending on the type of wound and progression of current wound healing. For example, in some embodiments, the methods of wound treatment of the present disclosure can utilize only the first stage to assist in the hemostasis phase of healing. In some embodiments, the methods of wound treatment of the present disclosure can utilize only the second stage to assist in the inflammatory phase of healing. In some embodiments, the methods of wound treatment of the present disclosure can utilize only the third stage to assist in the proliferative phase of healing. In some embodiments, the methods of wound treatment of the present disclosure can utilize only the third stage to assist in the maturation phase or remodeling stage of healing.

In some embodiments, a first protease composition having one or more proteases can be applied to a wound as a first treatment (i.e., the start of wound treatment). In some embodiments, the first protease composition having one or more proteases can include a first protease. In some embodiments, the first protease composition having one or more proteases can include a combination of a first and second protease. In some embodiments, the first protease composition having one or more proteases can include a combination of a first, second and third protease. In some embodiments, the first protease composition having one or more proteases can be a combination of any number of different proteases.

In some embodiments, the application of the first protease composition can be utilized to treat the wound over a time frame from approximately 6 to 72 hours. In some embodiments, the initial wound treatment can last for approximately 6 hours. In some embodiments, the initial wound treatment can last for approximately 12 hours. In some embodiments, the initial wound treatment can last for approximately 24 hours. In some embodiments, the initial wound treatment can last for approximately 72 hours or more.

The hemostasis stage is the first stage in the overlapping stages of wound healing and typically occurs immediately after injury. It is a rapid process that aims to stop bleeding and prevent further damage to the surrounding tissues. Hemostasis may last only a few minutes to several hours, depending on the severity and size of the wound. The duration of this stage also depends on factors such as the individual's overall health, the location of the wound, and the nature of the injury. Once hemostasis is achieved, the body can move onto the next stage of wound healing, which is inflammation.

The first stage of wound healing is known as the inflammatory phase, and it typically lasts for about 3-5 days after the initial injury. During this stage, the body responds to the injury by sending white blood cells to the site of the wound to begin the process of cleaning up any debris and bacteria, as well as to start the formation of new tissue. This stage is essential for setting the foundation for the later stages of wound healing.

After the first treatment, in some embodiments, the wound treatment methods of the present disclosure include applying a second protease composition having one or more proteases to the wound. In some embodiments, the second protease composition having one or more proteases can include a first protease. In some embodiments, the second protease composition having one or more proteases can include a combination of a first and second protease. In some embodiments, the second protease composition having one or more proteases can include a combination of a first, second and third protease. In some embodiments, the second protease composition having one or more proteases can be a combination of any number of different proteases.

In some embodiments, the application of the second protease composition can be utilized to treat the wound over a time frame from approximately 6 to 72 hours. In some embodiments, the second wound treatment stage can last for approximately 6 hours. In some embodiments, the second wound treatment stage can last for approximately 12 hours. In some embodiments, the second wound treatment stage can last for approximately 24 hours. In some embodiments, the second wound treatment stage can last for approximately 72 hours or more.

After a second treatment stage, in some embodiments, the wound treatment methods of the present disclosure include applying a third protease composition having one or more proteases to the wound. In some embodiments, the third protease composition having one or more proteases can include a first protease. In some embodiments, the third protease composition having one or more proteases can include a combination of a first and second protease. In some embodiments, the third protease composition having one or more proteases can include a combination of a first, second and third protease. In some embodiments, the third protease composition having one or more proteases can be a combination of any number of different proteases.

In some embodiments, the application of the third protease composition can be utilized to treat the wound over a time frame from approximately 6 to 72 hours. In some embodiments, the third wound treatment stage can last for approximately 6 hours. In some embodiments, the third wound treatment stage can last for approximately 12 hours. In some embodiments, the third wound treatment stage can last for approximately 24 hours. In some embodiments, the third wound treatment stage can last for approximately 72 hours or more.

The third stage of wound healing is known as the proliferative phase and typically begins around the third or fourth day after the injury and can last for several weeks. During this stage, the body begins to rebuild the damaged tissue by producing new blood vessels, collagen, and other components necessary for healing. Cells called fibroblasts migrate to the site of the wound and begin producing a protein called collagen, which forms the foundation of the new tissue. Blood vessels also grow into the wound to provide oxygen and nutrients necessary for the healing process.

The proliferative phase is a critical stage of wound healing, as it is responsible for restoring the structural integrity of the tissue and repairing the damage caused by the injury. The duration of this stage can vary depending on the size and severity of the wound, as well as the individual's overall health. Factors such as age, nutrition, and certain medical conditions can also affect the duration and quality of the proliferative phase.

After a third treatment stage, in some embodiments, the wound treatment methods of the present disclosure include applying a fourth protease composition having one or more proteases to the wound. In some embodiments, the fourth protease composition having one or more proteases can include a first protease. In some embodiments, the fourth protease composition having one or more proteases can include a combination of a first and second protease. In some embodiments, the fourth protease composition having one or more proteases can include a combination of a first, second and third protease. In some embodiments, the fourth protease composition having one or more proteases can be a combination of any number of different proteases.

In some embodiments, the application of the fourth composition can be utilized to treat the wound over a time frame from approximately 6 to 72 hours. In some embodiments, the fourth wound treatment stage can last for approximately 6 hours. In some embodiments, the fourth wound treatment stage can last for approximately 12 hours. In some embodiments, the fourth wound treatment stage can last for approximately 24 hours. In some embodiments, the fourth wound treatment stage can last for approximately 72 hours or more.

In some embodiments, the first, second, third and fourth treatment stages can include a time ranging from approximately 4 to 6 weeks. In some embodiments, various stages of treatment can overlap with one another. In some embodiments, various stages can be skipped or utilized multiple times to assist in wound healing. For example, the first, second, third or fourth treatment stage can be omitted or extended to subsequent stages. Additionally, the first, second, third and fourth stages can each be repeated, that is, the first, second, third or fourth protease composition can be applied any number of times during wound treatment. While various treatment stages have been described, a person of ordinary skill in the art can recognize that these stages can be continuous and overlapping stages of wound healing. For example, optimal wound healing can involve four continuous and overlapping stages that can include, for example the hemostasis phase, the inflammatory phase, the proliferative phase and the maturation phase or remodeling stage.

The fourth and final stage of wound healing is known as the remodeling or maturation phase, and it can begin as early as three weeks after the injury and continue for up to two years or more. During this stage, the newly formed tissue undergoes a process of maturation, where the collagen fibers that were laid down in the proliferative phase become stronger and more organized. The tissue also undergoes a process of remodeling, where excess collagen is removed, and the wound site is refined to restore the tissue's original structure and function.

The remodeling phase is essential for ensuring that the wound site becomes as strong and functional as possible, and the duration of this stage can vary widely depending on the severity of the injury, the location of the wound, and the individual's overall health. Factors such as age, nutrition, and the presence of underlying medical conditions can also impact the duration and quality of the remodeling phase.

In some embodiments, time frames and proteases (or protease combinations) for each treatment stage can be selected can be selected based on various properties to modulate activity of wound-related proteins, such as, MMPs, cytokines and growth factors, to thereby promote wound healing. As previously discussed, wound-related proteins can include, but are not limited to: MMPs, such as, for example, MMP-2, MMP-3, MMP-9; TIMPs, such as, for example, TIMP-1 and TIMP-2; TGF-α; interleukins, such as, for example, IL-β, IL-6, IL-10; growth factors, such as, for example, PDGF-AB, IGF-I, TGF-β, EGF, FGF basic, G-CSF, GM-CSF, VEGF; interferons, such as, for example, IFNα, IFNγ; CRP; and MIPs, such as, for example, MIP-1α, MIP-1β and MIP2.

Additionally, in some embodiments, time frames and proteases (or protease combinations) for each treatment stage can be selected can be selected based on various properties to modulate activity of at least one inflammatory or pro-inflammatory protein, to thereby prevent or treat inflammation. As described above, the inflammation-related proteins can include, but are not limited to: TNFα; IL-β. IL-6 and IL-10; serum amyloid A; fibrinogen; interferons, such as, for example IFNα and IFNγ; CRP; MIPs, such as, for example, MIP-1α, MIP-1β and MIP2; and MMPs, such as, for example, MMP-2, MMP-3 and MMP-9.

Additionally, the protease compositions can be included in the form of a kit. In some embodiments, the kit contains a wound-related protein or inflammation-related protein modulating protease composition comprising one or more proteases, a wound covering material, an antimicrobial composition, a wound wash, a wound spray, and/or a wound dressing.

In some embodiments, each of the one or more proteases is in a solution at a concentration of 10⁻⁸ to 2% (w/v). In some embodiments, the wound-related protein can include, without limitation, MMPs, MMP-2, MMP-3, MMP-9, TIMPs, TIMP TIMP-1, TIMP-2, TGF-α, interleukins, IL-β. IL-6, IL-10, growth factors, PDGF-AB, IGF-I, TGF-β, EGF, FGF basic, G-CSF, GM-CSF. VEGF, interferons, IFNα, IFNγ, CRP, MIPs, MIP-1α, MIP-1β, MIP2 and combinations thereof. In some embodiments, the inflammation-related protein can include, without limitation, TNFα, interleukins, such as, for example, IL-β, IL-6, IL-10, serum amyloid A, fibrinogen, interferons, IFNα, IFNγ, CRP, MIPs, MIP-1α, MIP-1β, MIP2, MMPs, MMP-2, MMP-3. MMP-9 and combinations thereof.

Moreover, the protease compositions and methods of use of the present disclosure can be used to reduce itching while healing wounds, and as such, are particularly beneficial for wound healing. The protease compositions of the present disclosure were evaluated on efficacy for itch treatment.

To evaluate the impact of five different lotions on “itchiness” of the skin as measured over a three-hour period after a single application to the legs of healthy, adult females with self-perceived dry and/or itchy legs.

This was a single-center, single-blind study to evaluate the impact of five different lotions on “itchiness” of the skin over a three-hour period. The study included one scheduled study visit where subjects were consented and had their eligibility reviewed. Eligible subjects were divided into three cells according to randomization.

Subjects assessed itching using a 100-millimeter visual analog scale at a baseline prior to treatment, immediately after application, and again three hours after application of the test articles to the lower legs. Following all three-hour procedures, subjects were dismissed from the study.

The study included healthy female subjects, 18 to 70 years of age, inclusively, fulfilling all of the inclusion and none of the exclusion criteria were eligible for participation in the study. The target was to complete sixty evaluable subjects. Actual enrollment was sixty-three subjects (21 subjects per cell), of which all completed the study.

Mean change in itchiness was compared to the baseline and the untreated control using the visual analog scale data. Within the treatment group, self-assessment values were significantly lower, relative to baseline, at the immediate time point. The untreated control self-assessment values were not significantly different, relative to baseline, at the immediate time point. The treatment group and the untreated control self-assessment values were significantly lower, relative to baseline, at the three-hour time point.

When comparing the change in subjects' self-assessment values, relative to baseline, across treatments there were no significant differences in subjects' self-assessment itch values between treatment group and the untreated control, regardless of time point. Overall, all test sites showed a significant improvement in itchiness three hours after test article application.

As such, various protease compositions of the present disclosure can further be utilized for itch relief, or to reduce itching during the treatment of wounds. In some embodiments, a protease composition including one or more proteases can be applied to an area of skin, for example, the legs, in order to relieve discomfort associated with irritated, dry or itchy skin. In some embodiments, the protease compositions can be applied on an as-needed basis or over an extended period of time.

In some embodiments, the protease compositions can include, without limitation, one or more proteases, an anionic surfactant (e.g., PROTEOL™ APL EF), glutamine, and niacinamide. In some embodiments, the protease compositions can include, without limitation, one or more proteases, glycerin, glutamine, proline, biotin, niacinamide, and sodium hyaluronate. In some embodiments, the protease compositions can include, without limitation, one or more proteases, myristyl nicotinate, glycerin, sodium hyaluronate, glutamine, proline, biotin, and niacinamide.

Although various embodiments of the present disclosure have been described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1. 1. 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

What is claimed is:

1. A method to promote tissue repair and wound healing, the method comprising:
applying a protease composition to an area of skin;
wherein the protease composition comprises one or more proteases;
modulating, by the protease composition, at least one of a wound-related protein or an inflammation-related protein; and
treating the area of skin for a prescribed period of time.

2. The method of claim 1, wherein the prescribed period of time is in a range of 6 to 72 hours.

3. The method of claim 1, wherein the one or more proteases is selected from the group consisting of acrosin, actinidain, ananain, asclepain, aspergillopepsin I, bacterial leucyl aminopeptidase, brachyurin, bromelain, calpain, carboxypeptidase A, caricain, cathepsin, chymopapain, chymosin, chymotrypsin, complement subcomponent C1r, cytosol aminopeptidase, DD-transpeptidase, dipeptidyl peptidase, deuterolysin, elastase, enteropeptidase, ficain, fragilysin, glycyl endopeptidase, hypodermin, ingensin, kallikrein, kininase, L-peptidase, methionine aminopeptidase, papain, pepsin, peptidyl-glycinamidase, plasmin, proproteinase, semenogelase, streptogrisin, subtilisin, thrombin and combinations thereof.

4. The method of claim 1, wherein the one or more proteases is in a solution at a concentration of 10⁻⁸ to 2% (w/v).

5. The method of claim 1, further comprising:
applying a second protease composition comprising at least one protease to the area of skin;
modulating, by the second protease composition, the at least one of the wound-related protein or the inflammation-related protein; and
treating the area of skin for a second prescribed period of time.

6. The method of claim 5, wherein the second prescribed period of time is in a range of 6 to 72 hours.

7. The method of claim 5, wherein the at least one protease is selected from the group consisting of acrosin, actinidain, ananain, asclepain, aspergillopepsin I, bacterial leucyl aminopeptidase, brachyurin, bromelain, calpain, carboxypeptidase A, caricain, cathepsin, chymopapain, chymosin, chymotrypsin, complement subcomponent C1r, cytosol aminopeptidase, DD-transpeptidase, dipeptidyl peptidase, deuterolysin, elastase, enteropeptidase, ficain, fragilysin, glycyl endopeptidase, hypodermin, ingensin, kallikrein, kininase, L-peptidase, methionine aminopeptidase, papain, pepsin, peptidyl-glycinamidase, plasmin, proproteinase, semenogelase, streptogrisin, subtilisin, thrombin and combinations thereof.

8. The method of claim 5, wherein the at least one protease is in a solution at a concentration of 10⁻⁸ to 2% (w/v).

9. The method of claim 1, further comprising:
applying a third protease composition comprising at least one protease to the area of skin;
modulating, by the third protease composition, the at least one of the wound-related protein or the inflammation-related protein; and
treating the area of skin for a third prescribed period of time.

10. The method of claim 9, wherein the third prescribed period of time is in a range of 6 to 72 hours.

11. The method of claim 9, the at least one protease is selected from the group consisting of acrosin, actinidain, ananain, asclepain, aspergillopepsin I, bacterial leucyl aminopeptidase, brachyurin, bromelain, calpain, carboxypeptidase A, caricain, cathepsin, chymopapain, chymosin, chymotrypsin, complement subcomponent C1r, cytosol aminopeptidase, DD-transpeptidase, dipeptidyl peptidase, deuterolysin, elastase, enteropeptidase, ficain, fragilysin, glycyl endopeptidase, hypodermin, ingensin, kallikrein, kininase, L-peptidase, methionine aminopeptidase, papain, pepsin, peptidyl-glycinamidase, plasmin, proproteinase, semenogelase, streptogrisin, subtilisin, thrombin and combinations thereof.

12. The method of claim 9, wherein the at least one protease is in a solution at a concentration of 10⁻⁸ to 2% (w/v).

13. The method of claim 1, further comprising:
applying a fourth protease composition comprising at least one protease to the area of skin;
modulating, by the fourth protease composition, the at least one of the wound-related protein or the inflammation-related protein; and
treating the area of skin for a fourth prescribed period of time.

14. The method of claim 13, wherein the fourth prescribed period of time is in a range of 6 to 72 hours.

15. The method of claim 13, wherein the at least one protease is selected from the group consisting of acrosin, actinidain, ananain, asclepain, aspergillopepsin I, bacterial leucyl aminopeptidase, brachyurin, bromelain, calpain, carboxypeptidase A, caricain, cathepsin, chymopapain, chymosin, chymotrypsin, complement subcomponent C1r, cytosol aminopeptidase, DD-transpeptidase, dipeptidyl peptidase, deuterolysin, elastase, enteropeptidase, ficain, fragilysin, glycyl endopeptidase, hypodermin, ingensin, kallikrein, kininase, L-peptidase, methionine aminopeptidase, papain, pepsin, peptidyl-glycinamidase, plasmin, proproteinase, semenogelase, streptogrisin, subtilisin, thrombin and combinations thereof.

16. The method of claim 13, wherein the at least one protease is in a solution at a concentration of 10⁻⁸ to 2% (w/v).

17. The method of claim 1, further comprising reducing itch-related symptoms at the area of skin.

18. The method of claim 1, wherein the wound-related protein is selected from the group consisting of matrix metalloproteinases (MMPs), matrix metalloproteinase-2 (MMP-2), matrix metalloproteinase-3 (MMP-3), matrix metalloproteinase-9 (MMP-9), tissue inhibitor metalloprotainases (TIMPs), TIMP metallopeptidase inhibitor 1 (TIMP-1), TIMP metallopeptidase inhibitor 2 (TIMP-2), transforming growth factor alpha (TGF-α), interleukins, interleukin-1ß (IL-β), interleukin-6 (IL-6), interleukin-10 (IL-10), growth factors, platelet-derived growth factor-AB (PDGF-AB), insulin-like growth factor I (IGF-I), transforming growth factor beta (TGF-β), epidermal growth factor (EGF), fibroblast growth factor (FGF) basic, granulocyte colony stimulating factor (G-CSF), granulocyte-macrophage colony-stimulating factor (GM-CSF), vascular endothelial growth factor (VEGF), interferons, interferon-α (IFNα), interferon-γ (IFNγ), C-reactive protein (CRP), macrophage inflammatory proteins (MIPs), macrophage inflammatory protein-1 alpha (MIP-1α), macrophage inflammatory protein-1 beta (MIP-1β), macrophage inflammatory protein-2 (MIP2) and combinations thereof.

19. The method of claim 1, wherein the inflammation-related protein is selected from the group consisting of TNFα, interleukins, such as, for example, IL-β, IL-6, IL-10, serum amyloid A, fibrinogen, interferons, IFNα, IFNγ, CRP, MIPs, MIP-1α, MIP-1β, MIP2, MMPs, MMP-2, MMP-3, MMP-9 and combinations thereof.

20. A kit to promote tissue repair and wound healing, the kit comprising:
a wound-related protein or inflammation-related protein modulating protease composition comprising one or more proteases;
a wound covering material;
wherein each of the one or more proteases is in a solution at a concentration of 10⁻⁸ to 2% (w/v);
wherein the wound-related protein is selected from the group consisting of matrix metalloproteinases (MMPs), matrix metalloproteinase-2 (MMP-2), matrix metalloproteinase-3 (MMP-3), matrix metalloproteinase-9 (MMP-9), tissue inhibitor metalloprotainases (TIMPs), TIMP metallopeptidase inhibitor 1 (TIMP-1), TIMP metallopeptidase inhibitor 2 (TIMP-2), transforming growth factor alpha (TGF-α), interleukins, interleukin-1β (IL-β), interleukin-6 (IL-6), interleukin-10 (IL-10), growth factors, platelet-derived growth factor-AB (PDGF-AB), insulin-like growth factor I (IGF-I), transforming growth factor beta (TGF-β), epidermal growth factor (EGF), fibroblast growth factor (FGF) basic, granulocyte colony stimulating factor (G-CSF), granulocyte-macrophage colony-stimulating factor (GM-CSF), vascular endothelial growth factor (VEGF), interferons, interferon-α (IFNα), interferon-γ (IFNγ), C-reactive protein (CRP), macrophage inflammatory proteins (MIPs), macrophage inflammatory protein-1 alpha (MIP-1α), macrophage inflammatory protein-1 beta (MIP-1β), macrophage inflammatory protein-2 (MIP2) and combinations thereof; and
wherein the inflammation-related protein is selected from the group consisting of TNFα, interleukins, such as, for example, IL-β, IL-6, IL-10, serum amyloid A, fibrinogen, interferons, IFNα, IFNγ, CRP, MIPs, MIP-1α, MIP-1β, MIP2, MMPs, MMP-2, MMP-3, MMP-9 and combinations thereof.
